(19) United States
(12) Patent Application Publication
(10) Pub. No.: US 2025/0303182 A1
(43) Pub. Date: Oct. 2, 2025

(54) SYSTEM AND METHOD OF DETECTING A LEAD ADAPTER

(71) Applicant: The Alfred E. Mann Foundation for Scientific Research, Valencia, CA (US)

(72) Inventors: Joseph L. Calderon, Santa Clarita, CA (US); Brian Michael Shelton, Ventura, CA (US); Neil Talbot, La Crescenta, CA (US)

(21) Appl. No.: 19/078,077

(22) Filed: Mar. 12, 2025

Related U.S. Application Data
(60) Provisional application No. 63/572,091, filed on Mar. 29, 2024.

(51) Int. Cl. A61N 1/375; A61N 1/08; A61N 1/372

(52) U.S. Cl. CPC A61N 1/3754; A61N 1/08; A61N 1/37247

(57) ABSTRACT

A step-down adapter for an implantable pulse generator includes a plug portion configured to plug into a receptacle of an implantable pulse generator. The plug portion includes electrical contacts, and at least two of the electrical contacts are shorted together. The step-down adapter also includes a receptacle portion connected to the plug portion. The receptacle portion includes electrical contacts, and the receptacle portion is configured to receive a proximal portion of an electrical stimulation lead.

### CROSS-REFERENCE TO RELATED APPLICATION(S)

The present application claims priority to and the benefit of U.S. Provisional Application No. 63/572,091, filed Mar. 29, 2024, the entire content of which is incorporated herein by reference.

### BACKGROUND

### 1. Field

The present disclosure relates to various embodiments of lead adapters for implantable pulse generators.

### 2. Description of the Related Art

Implantable pulse generators (IPGs) are utilized for a variety of therapeutic purposes, such as treating heart conditions, swallowing disorders onset by a stroke, sleep apnea, chronic back pain, and other medical conditions. IPGs are connected to one or more electrical stimulation leads to deliver electrical stimulation to a portion of the patient depending on the condition being treated. Additionally, related art IPGs may include a primary power supply that is not rechargeable. When an implanted IPG is at the end of its service life (e.g., due to a depleted or nearly depleted primary battery), the IPG may be explanted and a new IPG may be implanted in the patient. However, it may not be practical or possible to explant the electrical stimulation lead(s) connected to the IPG due to, for example, scar tissue formed around the electrical stimulation lead(s). Accordingly, it may not be practical or possible to implant new electrical stimulation lead(s) that are compatible with the new IPG implanted in the patient and the new IPG implanted in the patient may not be physically compatible with the electrical stimulation lead(s) already implanted in the patient.

The above information disclosed in this Background section is only to enhance understanding of background information pertaining to the present disclosure and may contain information that does not constitute prior art.

### SUMMARY

The present disclosure relates to various embodiments of a lead adapter for an implantable pulse generator. In one embodiment, the lead adapter is a step-down adapter including a plug or connector portion configured to plug into a receptacle of an implantable pulse generator. The plug portion includes electrical contacts and at least two of the electrical contacts are shorted together. The step-down adapter also includes a receptacle portion connected to the plug portion. The adapter receptacle portion includes electrical contacts and is configured to receive a proximal portion of an electrical stimulation lead.

A step-down adapter according to another embodiment of the present disclosure includes a plug or connector portion configured to plug into a receptacle of an implantable pulse generator. The plug portion includes electrical contacts and at least one resistor between two of the electrical contacts. The step-down adapter also includes a receptacle portion connected to the plug portion. The receptacle portion includes electrical contacts and is configured to receive a portion of an electrical stimulation lead.

The present disclosure also relates to various embodiments of a stimulator system. In one embodiment, the stimulator system includes an implantable pulse generator including a header, a lead receptacle in the header, electrical contacts in the lead receptacle, a processor, a non-volatile memory device, and a power supply. The system also includes a lead adapter configured to connect at least one electrical stimulation lead to the implantable pulse generator. The lead adapter includes a plug portion configured to extend into the lead receptacle in the header of the implantable pulse generator. The plug portion includes electrical contacts. The lead adapter also includes one receptacle portion connected to the plug portion. The one receptacle portion includes electrical contacts. The one receptacle portion is configured to receive a proximal end portion of the electrical stimulation lead. The non-volatile memory device includes instructions which, when executed by the processor, cause the implantable pulse generator to determine at least one of a resistance or an inductance between two of the electrical contacts of the plug portion and to determine a configuration i.e, (a) a step-down adapter (b) no-step down adapter used with the one electrical stimulation lead based on the resistance or the inductance.

The present disclosure also relates to various embodiments of a method of replacing an old implantable pulse generator implanted in a patient. In one embodiment, the method includes disconnecting at least one electrical stimulation lead implanted in the patient from the old implantable pulse generator, explanting the old implantable pulse generator, implanting a new implantable pulse generator, connecting the one electrical stimulation lead to the new implantable pulse generator with or without a step-down lead adapter, and detecting, by new implantable pulse generator, a configuration of the one electrical stimulation lead used with or without a step-down adapter.

This summary is provided to introduce a selection of features and concepts of embodiments of the present disclosure that are further described below in the detailed description. This summary is not intended to identify key or essential features of the claimed subject matter, nor is it intended to be used in limiting the scope of the claimed subject matter. One or more of the described features or tasks may be combined with one or more other described features or tasks to provide a workable device or method.

### BRIEF DESCRIPTION OF THE DRAWINGS

The features and advantages of embodiments of the present disclosure will become more apparent by reference to the following detailed description when considered in conjunction with the following drawings. In the drawings, like reference numerals are used throughout the figures to reference like features and components. The figures are not necessarily drawn to scale.

FIG. 1A is a schematic view of a stimulation system according to one embodiment of the present disclosure including an implantable pulse generator, an electrical stimulation lead, and a step-down adapter connecting the electrical stimulation lead to the implantable pulse generator;

FIG. 1B is a perspective view of the nerve cuff electrode having six electrode contacts within the cuff;

FIG. 2 is a block diagram of the embodiment of the implantable pulse generator illustrated in FIG. 1A;

FIG. 3A is a perspective view of a step-down adapter according to one embodiment of the present disclosure;

FIG. 3B is a perspective view of a step-down adapter according to another embodiment of the present disclosure;

FIG. 4 is a flowchart illustrating tasks of a method of replacing an implantable pulse generator according to one embodiment of the present disclosure.

### DETAILED DESCRIPTION

The present disclosure relates to various embodiments of a stimulation system including an implantable pulse generator (IPG), at least one stimulation lead having at least one electrode, and a lead adapter configured to connect the lead to the IPG.

In a conventional system configuration, a lead adapter is not used to connect the stimulation lead to the IPG if the IPG and its lead receptacle have been designed to be used with a stimulation lead having a compatible proximal connector. However, a lead adapter may be required if the IPG receptacle and stimulation lead connector are not compatible. At times, it may be necessary to use an IPG with more stimulation channels (i.e, having more electrical contacts in the IPG lead receptacle) than the number of electrode contacts in the stimulation lead. In that case, a step-down lead adapter is required to connect the IPG to the stimulation lead.

The present disclosure provides a design of a step-down lead adapter. In addition, the present disclosure provides a stimulation system that can automatically detect between at least two different stimulation system configurations including: (a) a conventional system with a stimulation lead and an IPG having the same number of stimulation channels, with or without a lead adapter, and (b) a system that has an IPG and a stimulation lead that uses a step-down lead adapter. Because the system can automatically detect the presence of the step-down adapter, the stimulation lead connected to this step-down adapter can be visually displayed on the screen on the clinician programmer and/or patient remote and thereby obviate the need for the clinician to determine and manually input the stimulation lead configuration. This saves clinician time and reduces potential error.

In some embodiments, two or more electrical contacts of the step-down lead adapter are shorted (shunted) and the IPG is configured to measure or determine the impedance through the electrode contacts and to utilize this information to detect or determine the configuration of the stimulation lead and/or the step-down lead adapter. In one or more embodiments, the lead adapter may include a controlled resistance, capacitance, or inductance between two or more of the electrode contacts of the lead adapter, and the IPG may be configured to measure or determine the resistance, inductance, or capacitance and to utilize this information to detect or determine the configuration of the stimulation lead and/or the step-down lead adapter. In this manner, the resistance, capacitance, or inductance of the lead contacts of the lead adapter encodes an identification (ID) used by the IPG to indicate the configuration of the lead adapter and/or the lead. The present disclosure also relates to various embodiments of a method of replacing an IPG implanted in a patient with another manufacturer's IPG (e.g., after the primary battery in the IPG is depleted). The method may include installing a step-down lead adapter to connect the existing stimulation lead if the newly implanted IPG has more stimulation channels compared to the existing lead. Sometimes, the IPG and stimulation lead may have the same number of stimulation channels, but because they are made by different manufacturers, the IPG and lead may be dimensionally incompatible. In that case, a lead adapter may be required between the new IPG and the old stimulation lead.

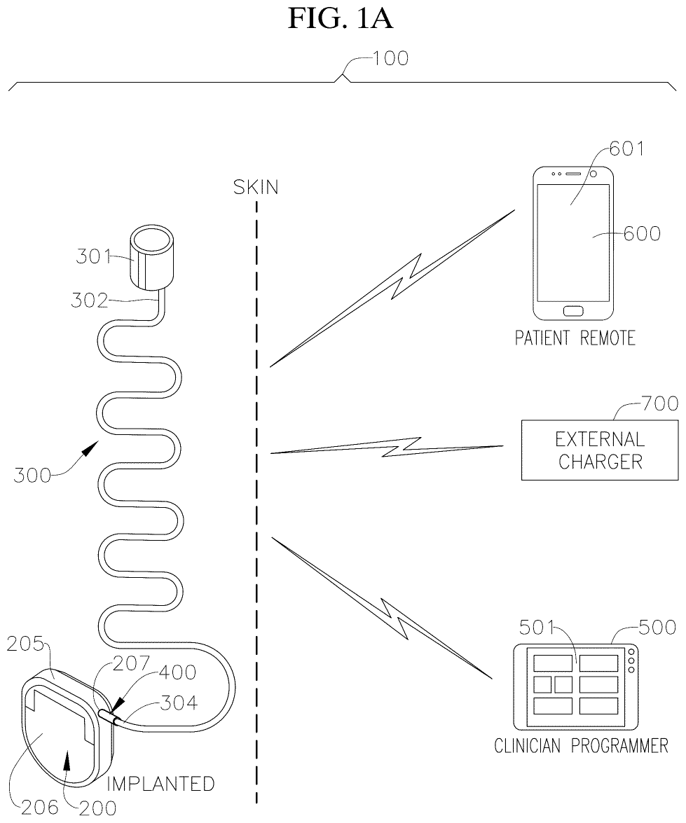

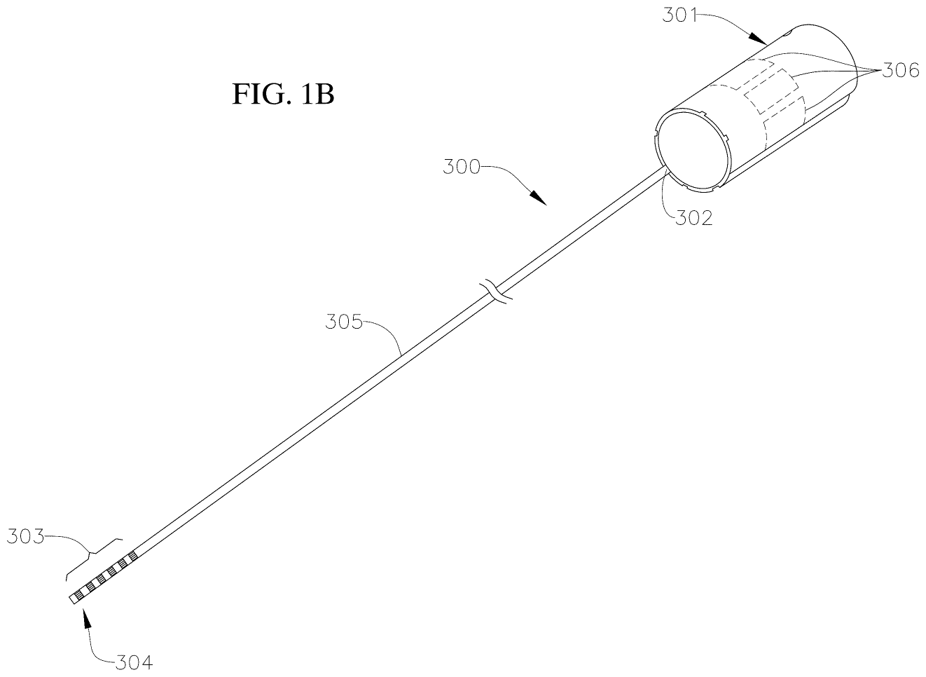

FIG. 1A shows an embodiment of a stimulation system 100 configured to treat a patient via electrical stimulation, such as neurostimulation, e.g., vagal nerve stimulation. According to one embodiment of the present disclosure the system includes an implantable pulse generator (IPG) 200, at least one electrical stimulation lead 300 having an electrode 301 (e.g., a nerve cuff) at a distal end 302 of the stimulation lead 300, and a lead adapter 400 configured to connect the electrical stimulation lead 300 to the IPG 200. The IPG has a header portion 205, typically made of epoxy, and a housing portion 206, typically made of a metal such as titanium alloy. The header portion 205 contains the lead receptacle 207. In one or more embodiments, the system 100 may also comprise external devices, such as a clinician programmer (CP) device 500 and/or a patient remote (PR) device 600 each electronically coupled to (i.e., in wireless RF communication with) the IPG 200. The lead adapter 400 is not needed in a conventional system if the stimulation lead 300 and the lead receptacle of the IPG 200 are compatible. A lead adapter as shown might be needed, however, if the stimulation lead 300 and IPG 200 are not dimensionally compatible. Adapter 400 can be a step-down adapter if the IPG 200 and the stimulation lead 300 have different numbers of stimulation channels (e.g., the IPG 200 has six channels contained in one lead receptacle, but the nerve cuff 301 only has two electrode contacts within the nerve cuff 301). The CP device 500 and the PR device 600 are each configured to bi-directionally communicate with the IPG 200 through the patient's skin (i.e., transcutaneously). The CP device 500 is configured to set one or more operating parameters or settings of the IPG 200. In one or more embodiments in which the IPG 200 is powered by a rechargeable battery instead of a single-use, primary cell battery, the stimulation system 100 may also include an external charger 700 configured to wirelessly (e.g., inductively) charge the IPG 200 through the patient's skin.

The electrical stimulation lead 300 includes an electrode 301 (e.g., a cuff electrode, a helical cuff electrode, a linear electrode, or a spinal cord paddle electrode having two or more electrode contacts) at the distal end 302 of the electrical stimulation lead 300 to periodically deliver an electric current pulse for a variety of therapeutic neurostimulation treatments for the patient. The type or kind of the electrode 301 may be selected based on the location and the type of nerves stimulated (e.g., a cuff electrode to stimulate a nerve bundle, such as the hypoglossal nerve or the vagus nerve; a linear lead to stimulate the brain, or a spinal cord lead, such as a paddle or a linear lead, to stimulate the spinal cord). The IPG 200 and the electrical stimulation lead 300 may be implanted in any suitable locations in the patient depending on the therapeutic treatment delivered by the system 100. In one or more embodiments, the IPG 200 may be implanted in a subcutaneous pocket in the upper chest of the patient, and the electrical stimulation lead 300 may extend from the IPG 200 via the neck to at least one of the patient's nerves, such as the vagus nerve, the hypoglossal nerve, or into the abdomen to the phrenic nerve which innervates the diaphragm.

The CP device 500 and the PR device 600 each include a display 501, 601 (e.g., a light-emitting diode (LED) display), respectively configured to display a graphical user interface (GUI). The GUI may display various information related to the stimulation mode (or stimulation parameters) of the IPG 200 and/or the configuration of the electrical stimulation lead 300, and the number of electrode contacts and their relative positions, among other information.

Referring to FIG. 1B, a stimulation lead 300, also in FIG. 1A, is shown in perspective view. The electrical stimulation lead 300 also includes a plurality of electrical contacts 303 at a proximal end 304 of a lead body 305 of the electrical stimulation lead 300. The electrical contacts 303 may include any suitably conductive metal, such as titanium or a stainless-steel alloy. In the illustrated embodiment, the electrode 301 is a nerve cuff electrode having a plurality of electrode contacts 306 inside the nerve cuff. The nerve cuff 301 (and the electrode contacts 306 therein) are at the distal end 302 of the lead body 305. In one or more embodiments, the electrode contacts 306 in the nerve cuff are made from a platinum-iridium alloy. In one or more embodiments, the stimulation lead 300 may include bilateral nerve cuffs.

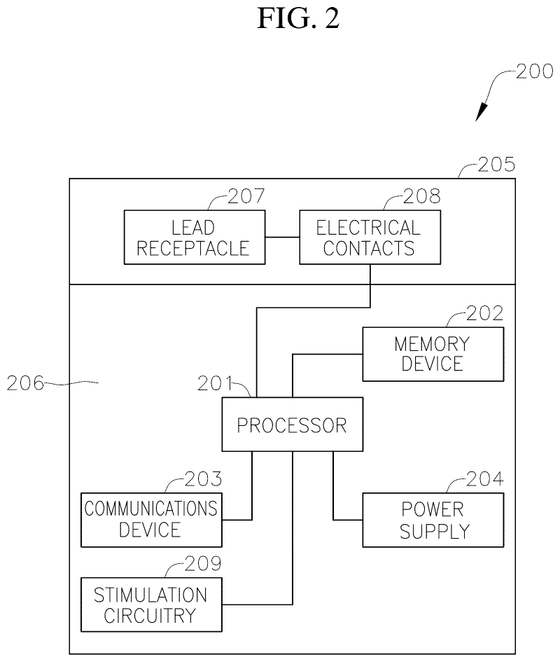

FIG. 2 shows a block diagram of an embodiment of the IPG 200. The IPG 200 includes a processor (e.g., a processing circuit) 201, a non-volatile memory device 202 (e.g., flash memory), a communications device 203 (e.g., a receiver and a transmitter, or a transceiver), and a power supply 204 (e.g., a primary battery or an inductively chargeable rechargeable battery). The communications device 203 provides wireless communication links through the skin of the patient to the CP device 500 and the PR device 600. Wireless links may include Bluetooth™, Bluetooth Low Energy or other protocols with suitable authentication and encryption to protect patient data. In one or more embodiments, the non-volatile memory device 202, the communications device 203, and the power supply 204 are in communication with each other over the processor 201. Additionally, in the illustrated embodiment, the processor 201, the non-volatile memory device 202, the communications device 203, and the power supply 204 are housed in a housing or a case 206. The case 206 may be a titanium alloy and may function as an indifferent, return anode. The case 206 includes a header 205 (e.g., a top epoxy part) and a lead receptacle 207 (e.g., a port or opening) in the header 205. The header 205 also includes a plurality of electrical contacts 208 (e.g., annular (ring-shaped) contacts) in the lead receptacle 207. The electrical contacts 208 may include any suitably conductive metal, such as titanium or a stainless-steel alloy.

In one or more embodiments, the electrical contacts 208 inside the lead receptacle 207 in the IPG header 205 are canted coil springs that are shaped into rings which accept the proximal connector end 304 of the stimulation lead 300. An example of such a canted coil spring connector is described in US Pat. No. 10,535,945, the entire content of which is incorporated herein by reference. In one embodiment, the header 205 of the IPG 200 includes six electrical contacts 208 in the lead receptacle 207, although in one or more embodiments the header 205 of the IPG 200 may include any other suitable number of electrical contacts 208 in the lead receptacle 207. The electrical contacts 208 are connected to dedicated circuitry 209 in the IPG 200 that provides stimulation pulses as controlled by the processor 201.

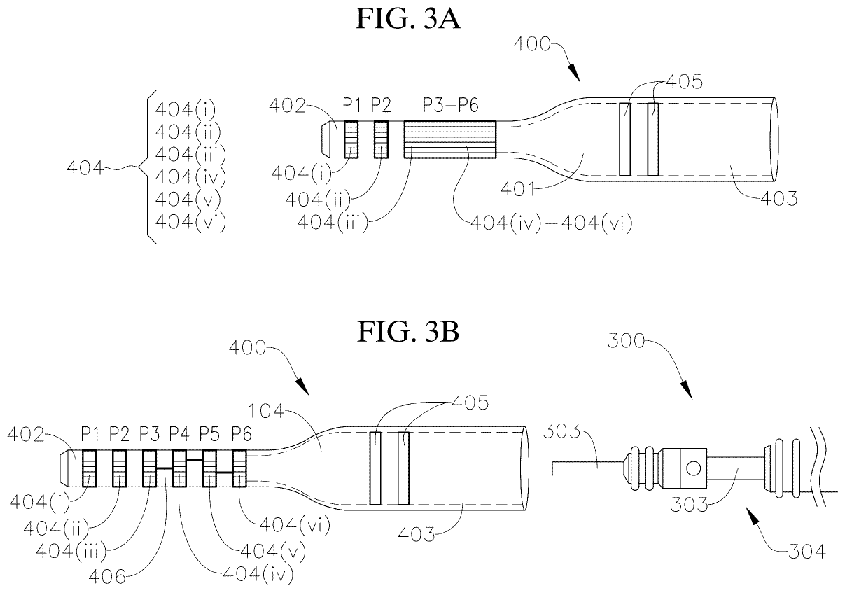

In one or more embodiments, the lead receptacle 207 may accommodate (e.g., receive) the proximal end 304 of the electrical stimulation lead 300. For instance, the lead receptacle 207 may accommodate (e.g., receive) the proximal end 304 of the electrical stimulation lead 300 in an embodiment in which the number of electrical contacts 303 at the proximal end 304 of the electrical stimulation lead 300 is equal to the number of electrical contacts 208 in the lead receptacle 207 of the IPG 200. However, in one or more embodiments, the lead receptacle 207 may accommodate (e.g., receive) the proximal portion of a lead adapter, for example, the step-down lead adapter 400 shown in FIG. 3B to connect the electrical stimulation lead 300 (FIG. 3B) to the IPG 200. For instance, the lead receptacle 207 may accommodate (e.g., receive) a portion of the lead adapter 400 in an embodiment in which the number of electrical contacts 303 (FIG. 3B) at the proximal end 304 of the electrical stimulation lead 300 (FIG. 3B) is different than the number of electrical contacts 208 in the lead receptacle 207 of the IPG 200. As described in more detail below, the number of electrical contacts 303 at the proximal end 304 of the electrical stimulation lead 300 may differ from the number of electrical contacts 208 in the lead receptacle 207 of the IPG 200 when the originally implanted IPG is at its end of life (e.g., the charge in the primary cell battery has been depleted or nearly depleted) and must be explanted from the patient and replaced with another IPG (e.g., a new IPG having a fully charged primary battery or a new IPG having a secondary battery that is rechargeable transcutaneously through the patient's skin). Typically, the electrical stimulation lead 300 cannot be explanted (e.g., due to scarring) and thus the electrical stimulation lead 300 cannot be replaced with a new electrical stimulation lead having a number of electrical contacts that matches the number of electrical contacts in the new IPG.

For clarity, as used in this disclosure, an IPG 200 with a six-channel stimulation system has a lead receptacle 207 with six electrical contacts 208. Each of these stimulation channels/contacts 208 may be independently selectable and programmable so that a corresponding electrically connected electrode contact 306 at the distal end 302 of the stimulation lead 300 can deliver a programmed stimulation current or voltage stimulus pulse as a cathode or act as a return anode. It will be understood that, in a bipolar stimulation mode, at least one of the electrode contacts 306 must be chosen as the return anode. Or in some embodiments, when the IPG metal portion of the housing 206 functions as a return, indifferent, anode, any one or more of the electrode contacts 306 in the stimulation lead 300 may function as a cathode. This latter mode of stimulation, where the IPG housing 206 functions as the return anode, is known as “monopolar” or “unipolar” stimulation.

The IPG 200 can be used in a number of different system configurations. As mentioned, the first system configuration is a conventional one where the IPG 200 is used with a compatible stimulation lead that is designed to be used together. For example, the IPG 200 may be a six-channel stimulation system with a single lead receptacle or port 207 which is connected to a stimulation lead with six electrical contacts at the proximal end of the lead and six electrode contacts at the distal end the stimulation lead. The distal end of the lead may be, for example, a linear electrode having an array of six electrode contacts or a nerve cuff electrode having six electrode contacts within the cuff. No adapter is required between the IPG and stimulation lead because the IPG receptacle in the header is designed specifically to be dimensionally compatible with the proximal connector end of the stimulation lead.

Another stimulation system configuration is one in which the IPG 200 is connected to a stimulation lead 300 that is from a different manufacturer than the manufacturer of the IPG 200, but having the same number of stimulation channels, i.e., same number of electrical contacts 208 in the IPG lead receptacle 207 and same number of electrode contacts 306 in the distal end 302 of the stimulation lead 300. In the cardiac pacing field, all manufacturers have agreed to a uniform connector standard for the cardiac pacemaker (IPG) and cardiac pacing lead, therefore customers may be able to mix and match a pacemaker and use it with a competitor pacing lead. Often, no adapter is needed between cardiac pacing leads and cardiac pacemakers when they are from different manufacturers. However, in the neuromodulation field, e.g., spinal cord stimulation, deep brain stimulation, vagus nerve stimulation, or hypoglossal nerve stimulation, there is no presently accepted lead connector standard that permits connection of IPGs of one manufacturer directly to the stimulation lead of another manufacturer. Typically, a lead adapter is, therefore, required between the IPG 200 and the stimulation lead 300 made by different manufacturers because of dimensional incompatibility in the IPG lead receptacle 207 and the proximal connector end 304 of the stimulation lead 300. Therefore, if a six-channel IPG 200 with a single lead receptacle or port 207 is to be connected to a stimulation lead 300, e.g., a nerve cuff, a paddle, or a linear lead, made from a different manufacturer, a lead adapter 400 must be used. When an IPG 200 is connected via a lead adapter 400 to a stimulation lead 300 with the same number of stimulation channels, i.e., electrode contacts, it will be functionally similar to the system where the stimulation lead 300 is connected directly to the IPG lead receptacle 207.

In another stimulation system configuration, an IPG having more stimulation channels may be replaced with an IPG having fewer stimulation channels. For example, a nerve cuff electrode having only two electrode contacts, one used as a cathode and another used as anode may be connected to an IPG having a single lead receptacle or port with six electrical contacts within the receptacle. If the IPG has a one-time-only use primary cell battery, and the battery becomes depleted or weak, the IPG must be replaced. The replacement IPG may also have a primary cell battery or it may have a rechargeable battery. It may be desired to use a replacement IPG from a different manufacturer than the manufacturer of the implanted stimulation lead and depleted IPG. However, if the replacement IPG has more electrical contacts (and more stimulation channels) than the stimulation lead, which has two electrode contacts, a specific type of lead adapter (a step-down lead adapter) can be used to connect the new IPG to the stimulation lead. This step-down lead adapter will only electrically connect two of the electrical connections in the IPG to the two electrode contacts in the stimulation lead. The step-down lead adapter can, however, be mechanically connected to all electrical contacts in the IPG receptacle, but the connections in the receptable that are not electrically connected to the stimulation lead will be either directly shorted or connected together by a resistor having a known value.

Although the clinician can determine what lead and/or adapter configuration is being used among at least these two stimulation system configurations: (1) the IPG used with a stimulation lead having the same number of stimulation channels/electrode contacts, with or without a lead adapter or (2) the IPG being used with a step-down lead adapter, where the IPG has more stimulation channels than electrode contacts on the lead, it is advantageous to have the IPG automatically detect the lead and/or adapter configuration.

While the clinician can determine what stimulation system configuration is being used by looking at the patient records, or in some cases, by physically feeling the presence of and the location of the stimulation lead and the adapter, if one is used, or by body scanning, e.g. X-rays, it is advantageous to have the IPG automatically detect among possible stimulation system configurations and to automatically and visually display the lead configuration to be programmed in the display of the clinician programmer and/or in the patient remote. This may be accomplished by using the disclosed design of the step-down lead adapter 400 and the designs of the IPG 200 and clinician programmer 500 and patient remote 600.

FIG. 3A shows lead adapter 400 according to one embodiment of the present disclosure, which is a step-down adapter meant to be used to connect a six-stimulation channel IPG 200 to a stimulation lead 300 that has two stimulation channels/two electrode contacts. The IPG 200 could use only bipolar stimulation, so that one electrode contact must be selected and programmed to be a return anode and another electrode contact is selected or programmed to be the cathode. In other embodiments, the IPG 200 may be configured to use the metal portion of the IPG housing 206 as the return, indifferent, anode and therefore configured to be in a monopolar stimulation mode. Then, in this monopolar mode, either or both of the electrode contacts 306 in the stimulation lead 300 may be selected as a cathode, or only one electrode contact 306 may be selected as the cathode and the other electrode contact 306 may be programmed to be turned off or made inactive. The step-down lead adapter 400 includes a body 401 having a plug portion 402 at a proximal end of the body 401 and a receptacle portion 403 at a distal end of the body 401. The plug portion 402 of the lead adapter 400 is configured to plug into the lead receptacle 207 in the IPG 200, and the receptacle portion 403 of the lead adapter 400 is configured to receive the proximal end 304 of the electrical stimulation lead 300.

In the illustrated embodiment, the lead adapter 400 includes a first plurality of electrical contacts 404 at the plug portion 402 (e.g., a first plurality of electrical contacts 404 exposed on an outer surface of the plug portion 402) and a second plurality of electrical contacts 405 inside the receptacle portion 403. As shown in FIG. 3B, in one or more embodiments, the number of electrical contacts 404 at the plug portion 402 is equal to the number of electrical contacts 208 in the lead receptacle 207 of the IPG 200. Although in the illustrated embodiment of FIG. 3B the lead adapter 400 includes six electrical contacts 404(i)-404(vi) (labeled P1, P2, P3, P4, P5, and P6), the lead adapter 400 may include any other suitable number of electrical contacts 404 depending on the number of electrical contacts 208 in the lead receptacle 207 of the IPG 200. When the plug portion 402 of the lead adapter 400 is plugged into the lead receptacle 207 of the IPG 200, the electrical contacts 404 of the lead adapter 400 contact the electrical contacts 208 in the lead receptacle 207 of the IPG 200. When the proximal end portion 304 of the electrical stimulation lead 300 is plugged into the receptacle portion 403 of the lead adapter 400, the electrical contacts 303 of the electrical stimulation lead 300 contact the electrical contacts 405 in the receptacle portion 403 of the lead adapter 400.

In the embodiment illustrated in FIG. 3A, the third, fourth, fifth, and sixth electrode contacts 404(iii)-404(vi) (labeled P3-P6) are shorted (shunted) together. In the illustrated embodiment, the third, fourth, fifth, and sixth electrode contacts 404(iii)-404(vi) include a single cylindrical contact, rather than individual (discrete) electrode contacts. The third, fourth, fifth, and sixth electrodes 404(iii)-404(vi) are “dummy” electrodes because they are not electrically connected to the electrical contacts 303 at the proximal end 304 of the electrical stimulation lead 300 (i.e., the third, fourth, fifth, and sixth electrode contacts 404(iii)-404(vi) are not configured to deliver electrical stimulation to the electrical contacts 303 of the electrical stimulation lead 300). Accordingly, in one or more embodiments, only the first and second electrodes 404(i) and 404(ii) are active and configured to deliver to deliver electrical stimulation to the electrical contacts 303 and the electrode(s) 301 of the electrical stimulation lead 300 (i.e., only the first and second electrodes 404(i) and 404(ii) among the six electrodes 404(i)-404(vi) are electrically connected to the electrical contacts 303 at the proximal end 304 of the electrical stimulation lead 300). In one or more embodiments, with examples shown in FIGS. 3A and 3B, any other of the electrical contacts 404 may be shorted (shunted) depending on the number of electrical contacts 303 at the proximal end 304 of the electrical stimulation lead 300. In one or more embodiments, the number of shorted electrical contacts 404 of the lead adapter 400 is equal to the difference between the number of electrical contacts 208 of the IPG 200 and the number of electrical contacts 303 at the proximal end 304 of the electrical stimulation lead 300 (e.g., in an embodiment in which the IPG 200 includes six electrical contacts 208 and the electrical stimulation lead 300 includes two electrical contacts 303, four of the electrical contacts 404 of the lead adapter 400 are shorted; in an embodiment in which the IPG 200 includes six electrical contacts 208 and the electrical stimulation lead 300 includes three electrical contacts 303, three of the electrical contacts 404 of the lead adapter 400 are shorted; and in an embodiment in which the IPG 200 includes five electrical contacts 208 and the electrical stimulation lead 300 includes two electrical contacts 303, three of the electrical contacts 404 of the lead adapter 400 are shorted).

In one or more embodiments, the non-volatile memory device 202 of the IPG 200 includes computer-readable instructions (e.g., software code) that, when executed by the processor 201, cause the IPG 200 to determine which electrode contacts 404 of the lead adapter 400 are shorted. For instance, in one or more embodiments, the instructions stored in the memory device 202, when executed by the processor 201, cause the IPG 200 to deliver current from the power supply 204 to the lead adapter 400 and to determine (e.g., measure or acquire) the impedance across the electrical contacts 404. Additionally, in one or more embodiments, the instructions, when executed by the processor 201, cause the IPG 200 to compare the measured impedance values to a threshold impedance value and to determine that those electrode contacts 404 that have an impedance value below the threshold impedance value are shorted (shunted). Additionally, in one or more embodiments, the non-volatile memory device 202 includes a lookup table associating different shorted electrodes 404 with different configurations of the electrical stimulation lead 300 (e.g., the lookup table may associate a short between the fifth electrode P5 and the sixth electrode P6 with a first lead configuration; a short between the fourth electrode P4 and the fifth electrode P5 with a second lead configuration; and a short between the fourth electrode P4, the fifth electrode P5 and the sixth electrode P6 with a third lead configuration). Accordingly, the manner in which the electrode contacts 404 are shorted encodes identification information regarding the configuration of the electrical stimulation lead 300.

Additionally, in one or more embodiments, the instructions stored in the memory device 202, when executed by the processor 201, cause the dedicated circuitry 209 of the IPG 200 to deliver stimulation to the electrodes 301 of the electrical stimulation lead 300 based on the configuration of the electrical stimulation lead 300 that was determined according to the manner in which the electrode contacts 404 of the lead adapter 400 were shorted. For example, in one or more embodiments, the instructions stored in the memory device 202, when executed by the processor 201, cause the IPG 200 to change the mode of stimulation based on the configuration of the electrical stimulation lead 300 that was determined according to the manner in which the electrode contacts 404 of the lead adapter 400 are shorted. Furthermore, in one or more embodiments, the instructions stored in the memory device 202, when executed by the processor 201, cause the communications device 203 of the IPG 200 to wirelessly transmit a signal to the CP device 500 and/or to the PR device 600 that causes a graphical user interface (GUI) displayed on the display 501, 601 of the CP device 500 and/or to the PR device 600 to display various information (e.g., the mode of operation of the IPG 200 and/or the configuration of the electrical stimulation lead 300) based on the manner in which the electrode contacts 404 of the lead adapter 400 are shorted.

FIG. 3B depicts an embodiment in which the lead adapter 400 includes at least one conductor wire (a short) or resistor 406 (e.g., a resistor having a fixed or known resistance) between the contacts 404. In the illustrated embodiment, the conductor wire or resistor 406 is connected between the fourth electrode contact 404(iv) and the fifth electrode contact 404(v), although in one or more embodiments the conductor wire or resistor 406 may be connected between any of the other electrode contacts 404, such as the fifth electrode 404(v) and the sixth electrode 404(vi).

In one or more embodiments, the non-volatile memory device 202 of the IPG 200 includes computer-readable instructions (e.g., software code) that, when executed by the processor 201, cause the IPG 200 to determine the resistance of the conductor wire or resistor 406 and/or the location of the resistor 406 (i.e., the electrical contacts 404 that are connected to the resistor 406). For instance, in one or more embodiments, the instructions stored in the memory device 202, when executed by the processor 201, cause the IPG 200 to deliver a constant (or substantially constant) current from the power supply 204 to the lead adapter 400 and to determine (e.g., measure or acquire) the voltage between the electrical contacts 404 and thereby determine (e.g., measure or acquire) the resistance of the conductor wire or resistor 406 and the location of the conductor wire or resistor 406 (i.e., the electrical contacts 404 that are connected to the conductor wire or resistor 406). Additionally, in one or more embodiments, the non-volatile memory device 202 includes a lookup table associating different resistance values and/or different locations of the resistor 406 with different configurations of the electrical stimulation lead 300 (e.g., the lookup table may associate a resistor 406 between the fifth electrode 404(v) (“P5”) and the sixth electrode 404(vi) (“P6”) with a first configuration of the electrical stimulation lead 300 and a resistor 406 between the fourth electrode 404(iv) (“P4”) and the fifth electrode 404(v) (“P5”) with a second configuration of the electrical stimulation lead 300). Accordingly, the resistance of the conductor wire or resistor 406 and/or the location of the conductor wire or resistor 406 between the electrical contacts 404 encodes identification information regarding the configuration of the electrical stimulation lead 300.

Additionally, in one or more embodiments, the instructions stored in the memory device 202, when executed by the processor 201, cause the dedicated circuitry 209 of the IPG 200 to deliver stimulation to the electrodes 301 of the electrical stimulation lead 300 based on the configuration of the electrical stimulation lead 300 that was determined according to the resistance of the conductor wire or resistor 406 and/or the location of the conductor wire or resistor 406 between the electrode contacts 404 of the lead adapter 400. For example, in one or more embodiments, the instructions stored in the memory device 202, when executed by the processor 201, cause the IPG 200 to change the mode of stimulation based on the configuration of the electrical stimulation lead 300 that was determined according to the resistance of the conductor wire or resistor 406 and/or the location of the conductor wire or resistor 406 between the electrode contacts 404 of the lead adapter 400. Furthermore, in one or more embodiments, the instructions stored in the memory device 202, when executed by the processor 201, cause the communications device 203 of the IPG 200 to wirelessly transmit a signal to the CP device 500 and/or to the PR device 600 that causes a graphical user interface (GUI) displayed on the display 501, 601 of the CP device 500 and/or to the PR device 600 to display various information (e.g., the mode of operation of the IPG 200 and/or the configuration of the electrical stimulation lead 300) based on the resistance of the conductor wire or resistor 406 and/or the location of the conductor wire or resistor 406 between the electrode contacts 404 of the lead adapter 400.

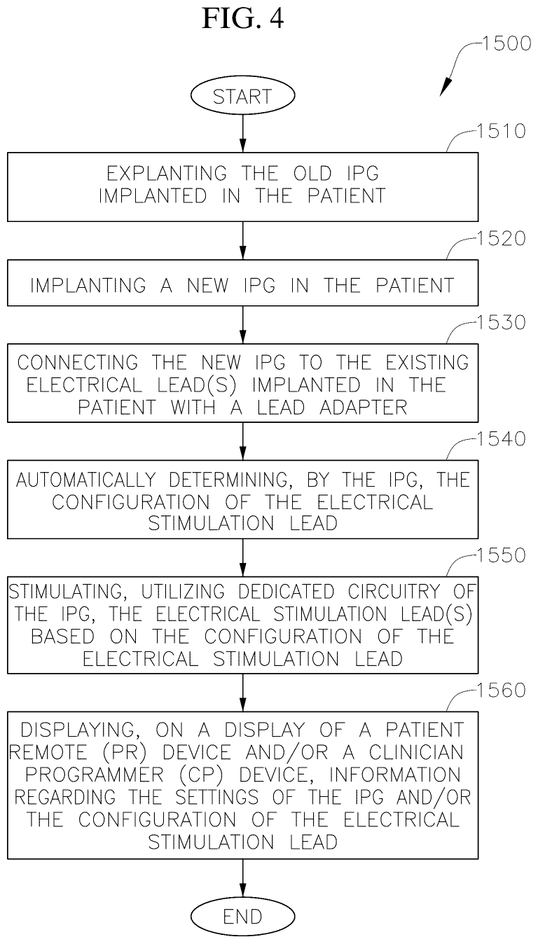

FIG. 4 is a flowchart illustrating tasks of a method 1500 of replacing an implantable pulse generator (IPG) implanted in a patient according to one embodiment of the present disclosure. The method 1500 may be utilized, for example, to replace an IPG implanted in a patient when the battery (e.g., the primary battery) has been depleted or is close to being depleted (e.g., the IPG is at or near the end of its useful lifecycle). In the illustrated embodiment, the method 1500 includes a task 1510 of removing (e.g., explanting) the old IPG implanted in the patient. The task 1510 of removing the old IPG includes detaching the one or more electrical leads connected to the old IPG.

In the illustrated embodiment, the method 1500 also includes a task 1520 of implanting a new IPG in the patient. The new IPG implanted in task 1520 may have a different configuration than the old IPG that was explanted from the patient in task 1510 (e.g., the new IPG implanted in task 1520 may be manufactured by a different manufacturer than the manufacturer of the IPG that was removed in task 1510). Additionally, as described above, it may not be practical or possible to explant the electrical stimulation lead from the patient due to, for example, scarring in the patient around the electrical stimulation lead. Accordingly, in one or more embodiments, the method may include not explanting the electrical stimulation lead from the patient and the configuration of the new IPG implanted in task 1520 may be physically incompatible with the electrical stimulation lead implanted in the patient. For instance, the lead receptacle of the new IPG may have a number of electrical contacts that differs (e.g., is greater than) from the number of electrical contacts at the proximal end of the electrical stimulation lead.

Accordingly, in an embodiment in which the configuration of the new IPG is incompatible with the electrical stimulation lead, the method 1500 includes a task 1530 of connecting the new IPG (implanted in task 1520) to the existing electrical stimulation lead(s) implanted in the patient with a lead adapter. In one or more embodiments, the lead adapter may be a step-down adapter (e.g., the lead adapter may have the same configuration as one of the lead adapters described above with reference to FIGS. 3A-3B).

In the illustrated embodiment, the method 1500 also includes a task 1540 of determining (e.g., automatically determining), by the IPG, the configuration of the electrical stimulation lead(s) that was attached to the IPG by the lead adapter in task 1530 if a step-down adapter is in fact used. In one or more embodiments, the task 1540 includes delivering current from the IPG to the lead adapter, determining (e.g., measuring or calculating) the impedance across the electrical contacts on the plug portion of the lead adapter, comparing the measured impedance values to a threshold impedance value, and determining that those electrode contacts that have an impedance value below the threshold impedance value are shorted (shunted). If, in task 1540, there is no short detected or no known resistance is detected between channels, then the method determines or concludes that the IPG is attached to a stimulation lead (with or without an adapter) with the same number of stimulation channels. In one or more embodiments, the task 1540 also includes referencing a lookup table (e.g., stored in the non-volatile memory device of the IPG), which associates different shorted electrodes with different configurations of the electrical stimulation lead, to determine the configuration of the electrical stimulation lead. In one or more embodiments, the task 1540 includes delivering a constant (or substantially constant) current from the IPG to the lead adapter and determining (e.g., measuring or acquiring) the voltage between the electrical contacts on the plug portion of the lead adapter and thereby determine (e.g., measure or acquire) the resistance and location of a resistor connected between two of the electrical contacts of the lead adapter. In one or more embodiments, the task 1540 also includes referencing a lookup table (e.g., stored in the non-volatile memory device of the IPG), which associates different resistance values and/or different locations of the resistor with different configurations of the electrical stimulation lead, to determine the configuration of the electrical stimulation lead. In one or more embodiments, the task 1540 may determine that the new IPG is connected to a (1) single electrical stimulation lead connected conventionally, e.g., an IPG with K number of stimulation channels connected to a lead with K number of stimulation channels, with or without a lead adapter or (2) a step-down lead adapter is being employed.

In the illustrated embodiment, the method 1500 also includes a task 1550 of stimulating, utilizing dedicated circuitry of the IPG, the electrical stimulation lead based on the configuration of the electrical stimulation lead determined in task 1540 (e.g., setting a stimulation mode, i.e., no step-down lead adapter or step-down lead adapter, of the IPG based on the configuration of the electrical stimulation lead determined in task 1540).

In one or more embodiments, the method 1500 may include a task 1560 of displaying, on a display of a patient remote (PR) device and/or a clinician programmer (CP) device in wireless communication with the new IPG, information regarding the operating mode or settings of the IPG and/or the configuration of the electrical stimulation lead(s), including the number of electrode contacts and their relative positions, as determined in task 1540. The task 1560 may include transmitting a wireless signal, from the new IPG to the PR device and/or the CP device, which cause a graphical user interface (GUI) displayed on the display of the PR device and/or the CP device to display information regarding the mode or settings of the new IPG and/or the configuration of the electrical stimulation lead(s). In the manner described above, the method 1500 enables the new IPG to function with the existing electrical stimulation lead(s) implanted in the patient, even when the electrical stimulation lead(s) is/are dimensionally incompatible with the new IPG and/or differ in the number of available stimulation channels.

The implantable pulse generator and/or any other relevant devices or components according to embodiments of the present invention described herein may be implemented utilizing any suitable hardware, firmware (e.g. an application-specific integrated circuit), software, or a combination of software, firmware, and hardware. For example, the various components of the device may be formed on one integrated circuit (IC) chip or on separate IC chips. Further, the various components of the device may be implemented on a flexible printed circuit film, a tape carrier package (TCP), a printed circuit board (PCB), or formed on one substrate. Further, the various components of the device may be a process or thread, running on one or more processors, in one or more computing devices, executing computer program instructions and interacting with other system components for performing the various functionalities described herein. The computer program instructions are stored in a memory which may be implemented in a computing device using a standard memory device, such as, for example, a random access memory (RAM). The computer program instructions may also be stored in other non-transitory computer readable media such as, for example, a CD-ROM, flash drive, or the like. Also, a person of skill in the art should recognize that the functionality of various computing devices may be combined or integrated into a single computing device, or the functionality of a particular computing device may be distributed across one or more other computing devices without departing from the scope of the exemplary embodiments of the present invention.

When a first element is described as being “coupled” or “connected” to a second element, the first element may be directly “coupled” or “connected” to the second element, or one or more other intervening elements may be located between the first element and the second element. As used herein, the singular forms “a”, “an” and “the” are intended to include the plural forms as well, unless the context clearly indicates otherwise. Further, the use of “may” when describing embodiments of the present disclosure refers to “one or more embodiments of the present disclosure.”

Although some embodiments of the present disclosure are disclosed herein, the present disclosure is not limited thereto, and the scope of the present disclosure is defined by the appended claims and equivalents thereof.

1 what is claimed is:

1. A step-down adapter for an implantable pulse generator, the step-down adapter comprising:
a plug portion configured to plug into a receptacle of the implantable pulse generator, the plug portion comprising a first plurality of electrical contacts, wherein at least two of the first plurality of electrical contacts are shorted together;
a receptacle portion connected to the plug portion, the receptacle portion comprising a second plurality of electrical contacts,
wherein the receptacle portion is configured to receive a proximal portion of an electrical stimulation lead.

2. The step-down adapter of claim 1, wherein a number of the second plurality of electrical contacts of the receptacle portion is less than a number of the first plurality of electrical contacts of the plug portion.

3. The step-down adapter of claim 2, wherein a number of the first plurality of electrical contacts that are shorted together is equal to a difference between the number of the first plurality of electrical contacts of the plug portion and the number of the second plurality of electrical contacts of the receptacle portion.

4. The step-down adapter of claim 3, wherein:
wherein the first plurality of electrical contacts of the plug portion comprises six electrical contacts,
wherein the second plurality of electrical contacts of the receptacle portion comprises two electrical contacts, and
wherein a second electrical contact, a third electrical contact, a fourth electrical contact, a fifth electrical contact, and a sixth electrical contact of the first plurality of electrical contacts of the plug portion are shorted to each other.

5. A step-down adapter for an implantable pulse generator, the step-down adapter comprising:
a plug portion configured to plug into a receptacle of the implantable pulse generator, the plug portion comprising a first plurality of electrical contacts and at least one resistor between two of the first plurality of electrical contacts; and
a receptacle portion connected to the plug portion, the receptacle portion comprising a second plurality of electrical contacts, wherein the receptacle portion is configured to receive a portion of an electrical stimulation lead.

6. The step-down adapter of claim 5, wherein the at least one resistor has a fixed resistance value.

7. The step-down adapter of claim 5, wherein a number of the second plurality of electrical contacts of the receptacle portion is less than a number of the first plurality of electrical contacts of the plug portion.

8. A stimulator system comprising:
an implantable pulse generator comprising:
a header;
a lead receptacle in the header;
a plurality of electrical contacts in the lead receptacle;
a processor;
a non-volatile memory device; and
a power supply;
a lead adapter configured to connect at least one electrical stimulation lead to the implantable pulse generator, the lead adapter comprising:
a plug portion configured to extend into the lead receptacle in the header of the implantable pulse generator, the plug portion comprising a first plurality of electrical contacts; and
at least one receptacle portion connected to the plug portion, the at least one receptacle portion comprising a second plurality of electrical contacts, wherein the at least one receptacle portion is configured to receive a proximal end portion of the electrical stimulation lead,
wherein the non-volatile memory device comprises instructions which, when executed by the processor, cause the implantable pulse generator to determine at least one of a resistance or an inductance between two of the first plurality of electrical contacts of the plug portion and to determine a configuration of the at least one electrical stimulation lead based on the resistance or the inductance.

9. The stimulator system of claim 8, wherein the lead adapter is a step-down adapter.

10. The stimulator system of claim 8, wherein a pair of electrical contacts of the first plurality of electrical contacts are shorted.

11. The stimulator system of claim 8, wherein the lead adapter further comprises at least one resistor between two of the first plurality of electrical contacts of the plug portion.

12. The stimulator system of claim 8, wherein the instructions stored in the non-volatile memory device, when executed by the processor, cause the implantable pulse generator to deliver electrical stimulation based on the configuration of the at least one electrical stimulation lead.

13. The stimulator system of claim 8, wherein:
the at least one receptacle portion comprises a single receptacle portion, and
the instructions stored in the non-volatile memory device, when executed by the processor, cause the implantable pulse generator to determine that the configuration of the at least one electrical stimulation lead is a single electrical stimulation lead.

14. The stimulator system of claim 8, further comprising a patient remote device in electronic communication with the implantable pulse generator, and wherein the instructions stored in the non-volatile memory device, when executed by the processor, cause the implantable pulse generator to transmit a signal to the patient remote device, the signal being configured to cause the patient remote device to display information based on the configuration of the at least one electrical stimulation lead.

15. The stimulator system of claim 8, further comprising a clinician programmer device in electronic communication with the implantable pulse generator, and wherein the instructions stored in the non-volatile memory device, when executed by the processor, cause the implantable pulse generator to transmit a signal to the clinician programmer device, the signal being configured to cause the clinician programmer device to display information based on the configuration of the at least one electrical stimulation lead.

16. A method of replacing an old implantable pulse generator implanted in a patient, the method comprising:
disconnecting at least one electrical stimulation lead implanted in the patient from the old implantable pulse generator;
explanting the old implantable pulse generator;
implanting a new implantable pulse generator;
connecting the at least one electrical stimulation lead to the new implantable pulse generator with a lead adapter comprising a plurality of electrical contacts; and
detecting, by new implantable pulse generator, a configuration of the at least one electrical stimulation lead.

17. The method of claim 16, wherein the connecting the at least one electrical stimulation lead to the new implantable pulse generator with the lead adapter comprises:
inserting a plug portion of the lead adapter into a lead receptacle in the new implantable pulse generator, the plug portion comprising a first plurality of electrical contacts; and
inserting a proximal end of the at least one electrical stimulation lead into a receptacle portion of the lead adapter, the receptacle portion comprising a second plurality of electrical contacts.

18. The method of claim 16, wherein the detecting the configuration of the at least one electrical stimulation lead comprises:
delivering current from the new implantable pulse generator to the lead adapter;
determining an impedance between at least two of the plurality of electrical contacts; and
comparing the impedance to a threshold impedance.

19. The method of claim 16, wherein the detecting the configuration of the at least one electrical stimulation lead comprises:
delivering current from the new implantable pulse generator to the lead adapter;
determining a resistance between at least two of the plurality of electrical contacts; and
comparing the resistance to a threshold resistance.

20. The method of claim 16, further comprising providing electrical stimulation, from the new implantable pulse generator to the at least one electrical stimulation lead, based on the configuration of the at least one electrical stimulation lead.

21. The method of claim 16, further comprising displaying, on a display of a patient remote device in wireless communication with the new implantable pulse generator, information based on the configuration of the at least one electrical stimulation lead.

22. The method of claim 16, further comprising displaying, on a display of a clinician programmer device in wireless communication with the new implantable pulse generator, information based on the configuration of the at least one electrical stimulation lead.
